(19) United States
(12) Patent Application Publication
(10) Pub. No.: US 2006/0064054 A1
(43) Pub. Date: Mar. 23, 2006

(54) Longitudinal sheath enforcement

(75) Inventors: Ghassan Sakakine, Rancho Santa Margarita, CA (US); Thomas DeMarchi, Mission Viejo, CA (US); Boun Pravong, Corona, CA (US); Kennii Pravongviengkham, Garden Grove, CA (US); John R. Brustad, Dana Point, CA (US); Charles C. Hart, Summerville, SC (US); Nabil Hilal, Laguna Niguel, CA (US)

Correspondence Address: APPLIED MEDICAL RESOUCES CORPORATION, 22872 Avenida Empresa, Rancho Santa Margarita, CA, 92688, US

(73) Assignee: Applied Medical Resources Corporation

(21) Appl. No.: 11/263,876

(22) Filed: Nov. 1, 2005

Related U.S. Application Data
(63) Continuation-in-part of application No. 11/152,945, filed on Jun. 14, 2005.
Application No. 11/152,945 is a continuation-in-part of application No. 10/832,867, filed on Apr. 26, 2004.
Application No. 11/152,945 is a continuation-in-part of application No. 10/766,138, filed on Jan. 28, 2004.
Application No. 10/832,867 is a continuation-in-part of application No. 10/298,116, filed on Nov. 15, 2002.
(60) Provisional application No. 60/624,246, filed on Nov. 1, 2004.
(60) Provisional application No. 60/465,310, filed on Apr. 25, 2003.

(51) Int. Cl. A61M 31/00

(52) U.S. Cl. 604/95.04; 604/528; 156/175

(57) ABSTRACT

A surgical access sheath with an elongate body and a lumen through the body is provided. The sheath has at least one longitudinal and radially spaced reinforcing wire longitudinally extending along the elongated body.

### CROSS-REFERENCE TO RELATED APPLICATION

This application is a continuation-in-part of U.S. patent application Ser. No. 11/152,945, filed Jun. 14, 2005 and claims benefit of U.S. Provisional Application No. 60/624,246, filed Nov. 1, 2004, the entire disclosures of which are hereby incorporated by reference as if set forth in full herein. U.S. patent application Ser. No. 11/152,945 is a continuation-in-part of U.S. patent application Ser. No. 10/832,867, filed Apr. 26, 2004, Ser. No. 10/766,138, filed Jan. 28, 2004 and Ser. No. 10/298,116, filed Nov. 15, 2002, and claims benefit of U.S. Provisional Application No. 60/579,500, filed Jun. 14, 2004, the entire disclosures of which are hereby incorporated by reference as if set forth in full herein.

### BACKGROUND

The present invention generally relates to surgical instruments or devices and, more specifically, to thin walled tubes, e.g., sheaths, shafts, cannulas, etc., that are enforced or reinforced along a longitudinal axis and resistant to stretching or elongation.

Thin walled tubes provide flexibility and a small overall size that permits these tubes to be applicable in many different surgical or medical instruments, e.g., access sheaths, cannulas, steerable/deflectable sheaths or cannulas, actuation shafts, non-flexible, flexible and/or pre/re-formed, for clamps, appliers, scopes, etc., and various other generally cylindrical devices utilizing or used in conjunction with small or thin walled tubing. However, by providing such flexibility and reduced size, the tubes can be susceptible to stretching or undue elongation. For example, an access sheath inserted into a patient may be pushed or pulled or otherwise manipulated to properly position the device. Such manipulation induces a generally longitudinal force along the length of the sheath or tube that can cause the tube to stretch.

If the access sheath is constructed with a thin wall made of a plastic or rubber material, the sheath may bend or twist during use. This may result in potential damage as the sharp edge of the kinked sheath may allow an endoscope or other device to complicate the surgical procedure. Moreover, a bent or kinked sheath may be useless because it cannot communicate and it may not allow the passage of an instrument. Additionally, the access sheath may be stretched or undesirably elongated that may damage or reduce the usefulness and operability of the sheath. As such, there is a desire in the art for a steerable access sheath that is durable enough to provide sufficient strength and stiffness to be guided through a body cavity or tissue and, at the same time, be flexible enough to perform intricate manipulations through the body cavity or tissue.

### SUMMARY

In one aspect, a surgical access sheath comprises an elongate body and at least one longitudinal and radially spaced reinforcement. The elongate body has a proximal end and a distal end and a lumen through the body. The at least one longitudinal and radially spaced reinforcement extends substantially along an entire length of the elongated body and generally parallel to the lumen.

In one aspect, a surgical access sheath comprises a tube having a substantially rigid portion with a first diameter and a substantially flexible portion with a second diameter and extending from the substantially rigid portion. The first diameter is larger than the second diameter and the tube has a primary lumen extending through the tube and a pull wire connected to the flexible portion of the elongated body. A first reinforcement wire extends longitudinally parallel to the primary lumen and a second reinforcement wire disposed radially from the first reinforcement wire extends longitudinally parallel to the first reinforcement wire. A third reinforcement wire is disposed raidally from the first and second reinforcement wire and extends longitudinally parallel to the first reinforcement wire and has a flat surface adjacent to the tube and has a larger surface area than the first and second reinforcement wires. A connector has a distal end connected to the tube and a proximal end including a funnel-shaped portion. The pull wire extends through the connector from the distal end to the proximal end and an actuator connected to the pull wire.

In one aspect, a method of manufacturing a surgical access sheath comprises coupling at least one wire to a mandrel, the at least one wire extending generally parallel to a longitudinal axis of the of the mandrel, wrapping a co-extruded wire around the at least one wire and the mandrel, the co-extruded wire comprising a plastic material, and heating the wrapped co-extruded wire until the plastic material melts and bonds windings of the co-extruded wire forming a generally elongate body.

In one aspect, an surgical access sheath is provided with a tube having a proximal end, a distal tapered end and a lumen extending through the tube. The sheath also comprises reinforcement means, e.g., one or more flat or flexible wires, extending longitudinally along the tube and generally parallel to the lumen and having a column strength greater than the tube.

Many of the attendant features of this invention will be more readily appreciated as the same becomes better understood by reference to the following detailed description and considered in connection with the accompanying drawings in which like reference symbols designate like parts throughout.

### BRIEF DESCRIPTION OF THE DRAWINGS

FIG. 1 illustrates aspects of a tube or sheath in accordance with various aspects of the present invention;

FIG. 2 illustrates aspects of a sheath with an enforcing wire coupled to a mandrel in accordance with various aspects of the present invention;

FIG. 3 illustrates aspects of a sheath in accordance with various aspects of the present invention;

FIG. 4 illustrates aspects of a sheath with an enforcing wire being flat in accordance with various aspects of the present invention; and

FIG. 5 illustrates aspects of a sheath with an enforcing wire positioned between a first tube and a co-extruded wire in accordance with various aspects of the present invention.

### DETAILED DESCRIPTION

In accordance with various aspects of the present invention, a wire is introduced along a longitudinal axis of the tube to counteract or resist any undue elongation of the tube during use. The wire is strong enough to resist the longitudinal forces and flexible enough to allow the tube to flex or bend. In one aspect, the wire is made of stainless steel or Kevlar. The wire is embedded in the tube or elongate body. This minimizes or maintains the overall size or diameter of the tube. In one aspect, multiple wires are used on opposing sides of the tube or generally around the tube to further reinforce the column strength of the tube or having a column strength greater than the tube. In one aspect, a wire or a plurality of wires are provided to provide a “weak-side/strong-side” or “flexible-side/rigid-side” arrangement of the tube or portions of the tube that allows the tube to be predisposed to bending in the desired direction. As such, one wire may be less flexible than another wire included in the tube or a wire may be provided one side of the tube without having a corresponding wire on a generally opposed side of the tube. In one aspect, the wires are generally flat to further reduce the overall size, provide additional or enhanced column strength and to provide flexibility to direct or deflect a portion of the tube in one or more directions.

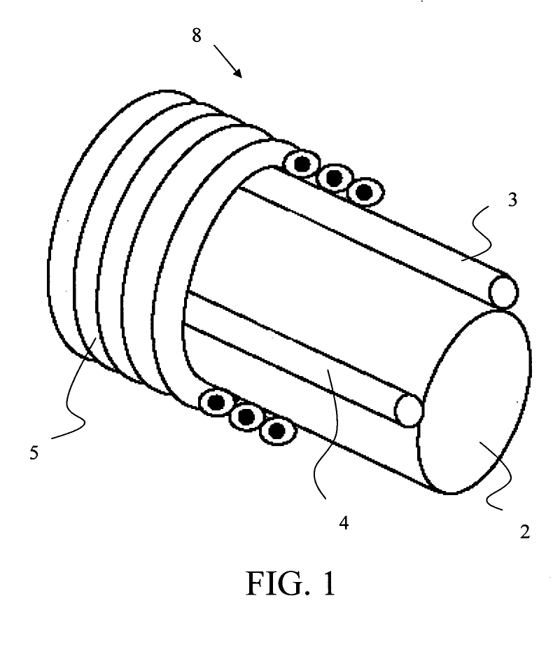

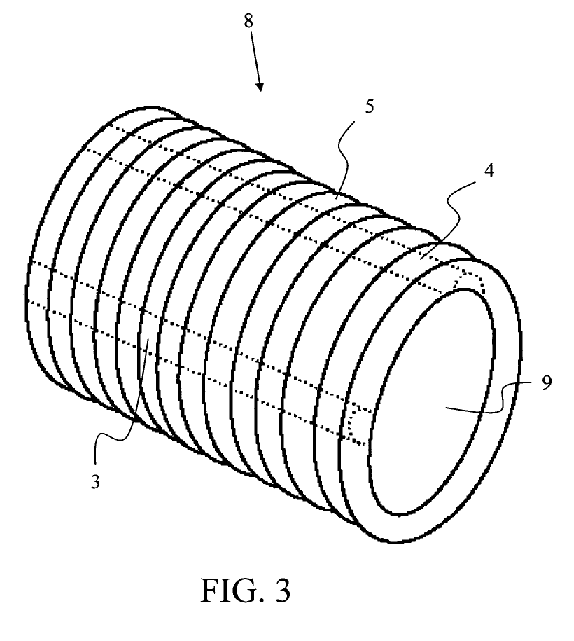

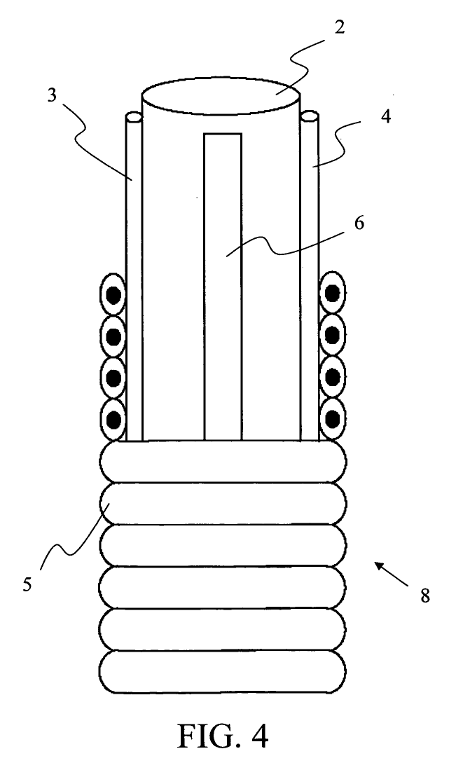

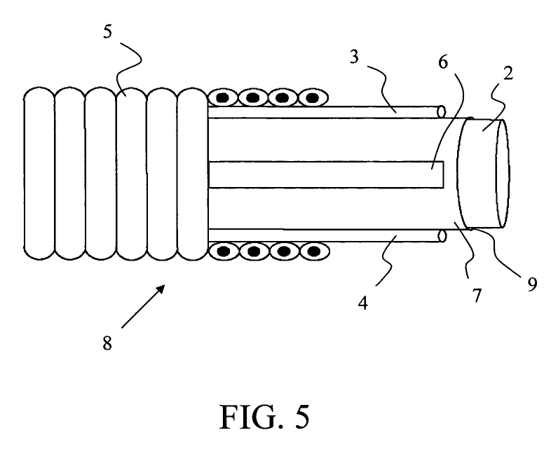

Referring now to FIGS. 1-5, various aspects of an access sheath or thin walled tube in accordance with various aspects of the prevent invention are shown. The thin walled tube or access sheath 8 is flexible and reinforced. In one aspect, the sheath 8 comprises a flexible material that is reinforced by one or more wires, e.g., wires 3, 4 and/or 6, extending along the longitudinal axis of the sheath 8. The one or more wires are embedded or otherwise secured to the tube 8. The wires are configured in different forms, configurations and comprise of or are made of various alloys, fabrics or any combination thereof. For example, one or all of the wires are a soft flexible wire, a flat wire, a pre/re-formable wire or any combination thereof. A soft flexible wire provides flexibility and also maintains column strength along the longitudinal axis of the tube. A flat wire, e.g., wire 6, extends longitudinally along the sheath and spreads along a radial portion of the sheath. A flat wire has an increased surface area extending along portions of the sheath and thus enhances and/or maintains column strength and provides flexibility or the ability to deflect the tube in various directions. A pre/re-forming wire can be formed or bent into a particular position or configuration and maintains that configuration, thereby enhancing the column strength of the sheath. As such, these reinforced tubes have added or enhanced pull strength or are resistant to elongation or axial or pulling forces that may be experienced by the tubes or sheaths during use.

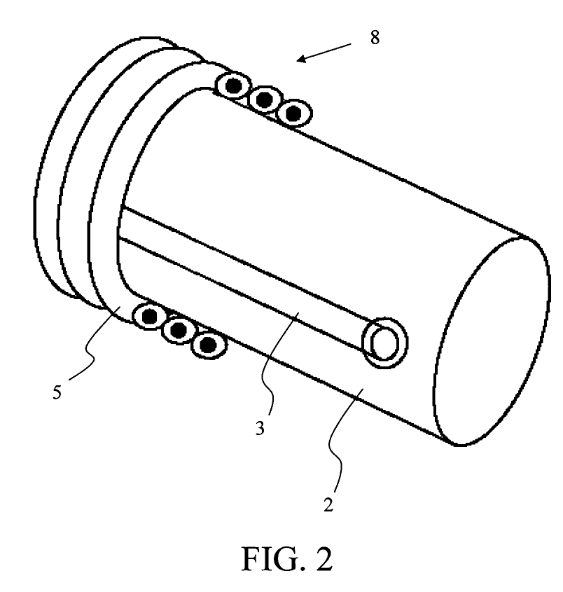

Referring to in FIG. 1, two wires 3 and 4 are placed on or positioned along a mandrel 2. The wires in one aspect extending along generally parallel to a longitudinal axis of the mandrel 2. The wires, in one aspect, are secured to the mandrel by threading it through holes or apertures at the end of the mandrel (FIG. 2). One wire may be more flexible than the other wire. With one wire more flexible or bendable than the other wire, the flexible wire 3, for example, can be used as or in conjunction with a tensioning device to deflect the tube.

In one aspect, a wire 5 is wound around a support member or mandrel 2 and thus the wires 3 and 4. The size and shape of mandrel generally defines the size and shape of a lumen of the access sheath or tube 8 and generally the profile of the tube. The mandrel 2, in one aspect, is stainless steel and made of or is coated with a low friction material or surface, e.g., Teflon or various mold releases, allowing for the mandrel to be easily removed from the tube 8. The mandrel is also substantially straight or tapered. The wire 5 is wound in an over counter fashion by using anchors or starting and stopping points substantially orthogonal of each other and thus winding the wire 5 in an oblique line along mandrel 2. As such, the wire 5 is wound such that the wire's tendency to unwind is counteracted. In one aspect, prior to the addition of the wire 5, the mandrel 2 is coated with or inserted into a plastic or PVC material tube to allow instruments and the like to be smoothly inserted into the lumen without interference from the wire 5.

The wire 5, in one aspect, is a plastic coated wire and particularly, a stainless steel co-extruded wire, coated with a plastic material in a co-extrusion process, with an approximate diameter of 0.006 inches fused, coated or otherwise included with a plastic material to make the total diameter of the wire 5 to be about 0.012 inches. The mandrel 2 along with wire 5 is placed into or inserted into a control tube. Air, in one aspect, is supplied, e.g., at 100 PSI, on the opposite end of insertion to assist insertion of the mandrel 2 by expanding the control tube. The control tube, in one aspect, may be made of silicon or a material with a higher melting point than the plastic coating of wire 5. This assembly is heated such that the plastic coating of wire 5 melts and adheres to itself to form a generally continuous tubular structure or elongate body or tube. As such, in one aspect, the plastic material of wire 5 melts and is formed above, below and in between the wire 5. Also, the plastic coating of wire 5 melts and adheres to the wires 3 and 4 to embed the wires into the continuous tubular structure 8.

In one aspect, the cross-section of the wires may be round, square, flat, or various other shapes and sizes to facilitate interlocking of the windings and wires. The placement, configuration and/or size or thickness of the wires 3 and 4 and/or the coating of the wire 5 is such that the embedding of the wires 3 and 4 does not interference with the continuous tubular structure 8, such as causing additional friction, protrusions or obstructions along the structure. Likewise, the overall size or dimensions of the tube are maintained or minimized. In one aspect, the wire 5 has voids, cavities or is generally flatten along a portion of the wire adjacent to the wires 3, 4 or 6. The control tube and the mandrel 2 are removed with the wires 3 and 4 disengaged from the mandrel 2. In one aspect, the plastic material is polyurethane, a thermoplastic, a thermoset or a plastic material having hard and/or soft durometer. The wire 5 is also in one aspect wound onto the mandrel in a multifilar fashion with material having alternating durometers.

In one aspect, a wire or wires, e.g., wires 3, 4 or 6, are placed between an extruded tube 7 and the wire 5, prior to having the wire 5 wound around the mandrel. As such, the wire is sandwiched between the extruded tube and the co-extruded wire. In one aspect, the tube 7 is formed similar to the tube 8 using a co-extruded wire. For example, a first co-extruded wire is wound onto a mandrel with one or more reinforcing wires are placed on or embedded along the first co-extruded wire and a second co-extruded wire is wound around the reinforcing wires and the first co-extruded wire. All of which are heated such that the first and second co-extruded wires melt and thereby adhere to form the generally continuous tubular structure or elongate body or tube.

In one aspect, a wire or wires, e.g., wires 3, 4 and 6, are embedded in an extruded tube 8. The wire or wires are introduced in the extrusion process in a portion of the die. The wires are configured to withstand the temperature/pressure utilized in the extrusion process to create the extruded tube. As a result, column strength of the extruded tube is increased while a continuous, smooth and thin tube is produced. In one aspect, the materials, hardness, pitch or shape of the wires vary depending on the surgical application. In one aspect, the sheath is coated or provided an outer layer to facilitate use, e.g., entry into a body cavity or through body conduits. For example, the sheath is dipped into a solvent solution to form an outer layer on the sheath. In one aspect, one or more of the wires 3, 4 or 6 are separately placed within a tube or within a second or secondary lumen of a second elongate body or tube formed by a wire similar to wire 5 wound around one or more of the reinforcing wires 3, 4 or 6. The secondary lumen as such is generally parallel to the first or primary lumen and has a diameter smaller than the diameter of the first lumen.

In one aspect, wire 3 is on one side of the tube and wire 4 is on an opposing or adjacent side of the tube both reinforcing the column strength of the tube. In one aspect, wire 4 or a portion of wire 4 is more flexible and/or weaker than wire 3. For example, wire 4 is made of or comprises of a flexible or more flexible material than the wire 3 or is weaken having voids or divots throughout the length or portions of the wire 4. As such, the side of the tube including wire 4 is predisposed to bending in a desired direction then the side of the tube including wire 3. Thus, using at least one of the wires or a separate tensioning device, such as a control or pull wire, the tube or portions of the tube may be steered or deflected. A tensioning device when acted upon, e.g., pulled, deflects the access sheath 8. As such, in one aspect, one or more pull wires are embedded in one or more reinforcing wires extending along the access sheath 8.

The one or more tensioning devices and/or one or more wires in one aspect are attached to an actuator, which is coupled or integrated with the access sheath 8. The actuator may include a handle, thumb-actuated knob, ring, or another type of device to manipulate or control the tensioning device. For example, a ring connected to a pull wire may be drawn proximally to provide tension to the tensioning device. When the ring is released, the pull wire moves distally to loosen tension or cause the tensioning device to loosen to allow the access sheath to straighten or return back to a previous or initial form. In one aspect, a connector is connected to the tube on one end and on the other end having a funnel-shaped portion. One or more tensioning devices, e.g., pull wires, extend through the connector and to an actuator connected thereto.

As such, by manipulating the actuator, a user can steer the access sheath 8 to navigate circuitous or torturous conduits or cavities within the body to access the surgical site or point of interest. The actuator in various aspects is configured to be in-line, offset or remote from the access sheath 8. Additionally, the access sheath may comprise a plurality of pull wires attached to a plurality of thumbwheels, axles, knobs or other types of movable components of an actuator or actuation hand-piece to deflect the access sheath in one or more different directions.

The access sheath 8 in one aspect has a lumen 9 extending through the sheath. The lumen 9 is sized and configured to provide an access pathway to a surgical site or a target site for the surgical procedure. For example, lumen 9 provides a conduit to advance a surgical instrument, e.g., a dilator, or diagnostic and therapeutic elements, e.g., a contrast agent, to the surgical or target site. As such, the access sheath via the lumen 9 provides a conduit or a channel from outside the body to the point of interest for the insertion and/or withdrawal of instruments, tissue or other items used for or in conjunction with the surgical procedure.

The forces or stress accumulated along the sheath that may cause kinks in the sheath can be distributed along the access sheath by the composite construction of the tube and/or are further counteracted by the reinforcing wire(s), e.g., wires 3, 4 and 6. Thus, kinks in the access sheath are reduced. The reinforcing wires in one aspect or thin and thus also allow the tube walls to be thin without reducing durability or strength in the sheath. Thus, the overall or outer diameter of the sheath is small, which also reduces the incision or insertion point for the sheath, without reducing the size or diameter of the lumen. As such, the access sheath of various aspects of the present invention has thin walled portions, a large lumen, an atraumatic end, and a kink resistant construction and is strong, stiff and yet flexible enough to be intricately guided through the body cavity or tissue.

In one aspect, manufacturing various aspects of the access sheath in accordance with various aspects of the present invention comprises attaching at least one reinforced wire to a mandrel, wrapping a first coated wire around a mandrel, and heating the reinforced wire, the mandrel, and the first wire. In aspect, the reinforcing wire may be a flattened member extending along the longitudinal axis of the mandrel. In one aspect, one or more reinforcing wires are introduced with the tube during extrusion to embed the wire into the tube. In one aspect, one or more reinforcing wires are sandwiched between an extruded tube and a co-extruded wire wound around the tube and the reinforcing wire. In yet another aspect, one or more reinforcing wires are embedded into an extruded tube. In one aspect, one or more reinforcing wires is a co-extruded wire or comprise a polymer or metallic, e.g., stainless steel, material and/or with a column strength greater than the column strength of the formed tube.

In one aspect, manufacturing various aspects of the access sheath in accordance with various aspects of the present invention comprises securing a first tubing, such as a polyurethane tubing, e.g., Pellethane 95AE, with a Kevlar wire, e.g., a reinforcement, integrated, extending, embedded, or threaded through the first tubing, to a mandrel. The first tubing is sized and configured to accommodate the size and configuration of a desired enforced tube, e.g., a 25 mm, 9 F tube. The first tubing is secured to the mandrel using adhesive or threading the tubing through apertures or slots in the mandrel. A co-extruded wire, e.g., 0.006 wire coated with 0.01 polyurethane (Pellethane) is wound around the first tubing using, for example, a lathe. The first tubing, co-extruded wire and mandrel are inserted into a second tube. Air, in one embodiment, is supplied, e.g., at 100 PSI, on the opposite end of insertion to assist insertion of the components by expanding the second tube. The second tube in one aspect acts as a control tube that maintains the components and associated materials in close proximity with each other to ensure a continuous and smooth tube is produced.

The first tubing, co-extruded wire, mandrel and second tube are heated. In one example, the components are heated for about 12 minutes for 176 degrees. The second tube, in one embodiment, may be made of silicon or a material with a higher melting point than the co-extruded wire. The mandrel and second tube are then removed. As a result, an enforced tube that is a continuous, smooth, small and thin tube having substantial column strength and flexibility is produced. In one aspect, the components are cooled and air is again supplied, e.g., at 100 PSI, at one end to assist in the removal of the second tube. The mandrel, in one embodiment, is removed by clamping one end, removing the secured point(s), twisting in a winding or unwinding direction and sliding the mandrel from the continuous tube.

Various other examples of processes that may be used to manufacture the sheath or portions of the sheath are described in U.S. patent application Ser. Nos. 10/766,138 and 10/298,116, the disclosures of which are hereby incorporated by reference. It is appreciated that these processes or portions of the processes may be varied or combined with the described process herein and vice versa. For example, various ring-shaped elements, such as, plastic rings, metallic rings, un-reinforced plastic rings and metal reinforced plastic rings, and the like may be utilized instead of or in addition to the coiled wires.

In one aspect of the present invention, various aspects of the sheaths previously described, are applicable to various thin walled tubes or cannulas in which the tube utilized in different devices, assemblies or applications. As such, these thin walled tubes may be reinforced along the longitudinal axis of the sheath in the manner previously described. It should be appreciated that the term wire used in the specification is not intended to limit or specify a particular type of composition or material of the element or wire.

Accordingly, various aspects of the present invention provide a longitudinal reinforced surgical access sheath. Although this invention has been described in certain specific embodiments, many additional modifications and variations would be apparent to those skilled in the art. It is therefore to be understood that this invention may be practiced otherwise than specifically described, including various changes in the size, shape and materials, without departing from the scope and spirit of the present invention. Thus, embodiments of the present invention should be considered in all respects as illustrative and not restrictive, the scope of the present invention to be determined by the appended claims and their equivalents rather than the foregoing description.

1. A surgical access sheath comprising:
an elongate body having a proximal end and a distal end, the body having a lumen through the body; and
at least one longitudinal and radially spaced reinforcement extending substantially along an entire length of the elongated body and generally parallel to the lumen.

2. The sheath of claim 1 further comprising:
an second elongate body having a second lumen extending substantially parallel to the lumen, having a diameter smaller than a diameter of the lumen and having the reinforcement extending through the second lumen; and
an actuator connected to the reinforcement.

3. The sheath of claim 1 further comprising:
a tensioning device;
an second elongate body having a second lumen extending substantially parallel to the lumen, having a diameter smaller than a diameter of the lumen and having the tensioning device extending through the second lumen; and
an actuator connected to the tensioning device distally from the proximal end of the elongated body to control tension of the tensioning device.

4. The sheath of claim 3 wherein the tensioning device is one of a pull wire and flexible flatten wire.

5. The sheath of claim 1 further comprising an actuator connected to the reinforcement, the reinforcement being a wire.

6. The sheath of claim 1 wherein the reinforcement comprises one of a flat wire and a co-extruded wire.

6. The sheath of claim 1 further comprising steerable regions disposed along non-adjacent portions of the elongate body.

7. The sheath of claim 1 wherein the distal end of the elongate body is tapered.

8. The sheath of claim 1 wherein the body comprises a heated co-extruded wire.

9. The sheath of claim 1 further comprising a second elongate body having the reinforcement disposed between the elongate body and the second elongate body, the second elongate body comprises of a plastic material.

10. A surgical access sheath comprising:
a tube having a substantially rigid portion having a first diameter and a substantially flexible portion having a second diameter and extending from the substantially rigid portion, the first diameter being larger than the second diameter, the tube having a primary lumen extending through the tube and a pull wire connected to the flexible portion of the elongated body;
a first reinforcement wire extending longitudinally parallel to the primary lumen;
a second reinforcement wire disposed radially from the first reinforcement wire and extending longitudinally parallel to the first reinforcement wire;
a third reinforcement wire disposed raidally from the first and second reinforcement wire and extending longitudinally parallel to the first reinforcement wire and having a flat surface adjacent to the tube and having a larger surface area than the first and second reinforcement wires;
a connector having a distal end connected to the tube and a proximal end with a funnel-shaped portion, the pull wire extending through the connector from the distal end to the proximal end; and
an actuator connected to the pull wire.

11. The sheath of claim 10 wherein the tube has a secondary lumen extending through the tube generally parallel to the primary lumen having a third diameter smaller than the second diameter, and the pull wire extends through the secondary lumen.

12. The sheath of claim 10 wherein the tube has a secondary lumen extending through the tube generally parallel to the primary lumen having a third diameter smaller than the second diameter, and one of the first and second reinforcement wire extends through the secondary lumen.

13. The sheath of claim 10 wherein one of the first, second, and third reinforcement wire is connected to the pull wire.

14. The sheath of claim 10 wherein the first reinforcing wire and the second reinforcing wire are radially disposed within the tube opposite of each other.

15. A method of manufacturing a surgical access sheath comprising:
coupling at least one wire to a mandrel, the at least one wire extending generally parallel to a longitudinal axis of the of the mandrel;
wrapping a co-extruded wire around the at least one wire and the mandrel, the co-extruded wire comprising a plastic material; and
heating the wrapped co-extruded wire until the plastic material melts and bonds windings of the co-extruded wire forming a generally elongate body.

16. The method of claim 15 wherein the co-extruded wire comprises at least one of polyurethane, a thermoplastic material and a thermoset material.

17. The method of claim 15 further comprising:
securing windings of the wrapped co-extruded wire within a shrink tube and heating the shrink tube until it encapsulates all of the windings; and
coupling an extruded tube to the mandrel.

18. The method of claim 15 further comprising dipping the sheath in a solvent based solution forming an outer layer of the sheath.

19. A surgical access sheath comprising:
a tube having a proximal end and a distal tapered end, the tube having a lumen extending through the tube; and
a reinforcement means extending longitudinally along the tube and generally parallel to the lumen and having a column strength greater than the tube.

20. The sheath of claim 19 wherein the reinforcement means comprises a plurality of wires extending longitudinally parallel to the primary lumen and substantially along an entire length of the tube, each wire disposed radially around the lumen.
